(19) United States
(12) Patent Application Publication
(10) Pub. No.: US 2002/0137100 A1
(43) Pub. Date: Sep. 26, 2002

(54) Method for determining the fertility of mammals, especially of humans

(75) Inventors: Karl Oskar Illmensee, Innsbruck (AT); Hans Dieplinger, Innsbruck (AT)

Correspondence Address: Mark B. Wilson, Fulbright Jaworski L.L.P., Suite 2400, 600 Congress Avenue, Austin, TX, 78701, US

(73) Assignee: Vitateq Biotechnology GMBH, Innsbruck (AT)

(21) Appl. No.: 10/104,918

(22) Filed: Mar. 22, 2002

Related U.S. Application Data
(63) Continuation of application No. PCT/AT00/00256, filed on Sep. 25, 2000.

(30) Foreign Application Priority Data
Sep. 23, 1999 (AT) A 1626/99

(51) Int. Cl. G01N 33/53

(52) U.S. Cl. 435/7.1

(57) ABSTRACT

Disclosed is a method for determining the fertility of mammals, in particular of humans, wherein

a body or organ fluid is taken from a mammal,

the leptin content is determined in this body or organ fluid, and

the determined leptin content is compared with a reference value so as to determine the fertility,

the use of leptin for determining the fertility of mammals, as well as a kit for carrying out this method.

The invention relates to a method of determining the fertility of mammals, in particular of humans.

Due to the increase in the knowledge and abilities concerning the in vitro-fertilization technique, the demand of reliable and also low-cost fertility determining methods has highly increased. What is involved is not merely the basic diagnosis of fertility disturbances as such, but increasingly also the detection of the degrees of fertility and the determination of the fertilization and/or implantation of the fertilized oocytes.

Leptin was discovered in 1994 as a central hunger-regulating metabolic hormone and associated with further multiple functions. Thus, in a series of tests the importance of this hormone could be shown for the reproduction of animals and humans, the function and regulation of leptin within the scope of reproduction not yet having been clarified in detail.

Leptin is a 16 kD protein of the cytokine family (Zhang Y. et al., Nature 1994, 372, pp. 425-432) and is synthesized and secreted by adipocytes (R. V. Considine et al., J. Clin. Invest 1995, 95, 2986-2988). Leptin circulates in blood and is bound to its soluble receptor (G. H. Lee et al., Nature 1996, 379, 632-635). A further receptor, OB-R_{S}, is responsible for the transportation of leptin across the blood-brain barrier (L. A. Tartaglia, Cell 1995, 83, 1263-1271). In the hunger center of the hypothalamus, leptin binds to its long, signal-transmitting receptor, OB-R_{L} (H. Chen et al., Cell 1996, 84, 491-495), whereby the production of the neuropeptide Y (NPY) is inhibited there (T. W. Stephens et al., Nature 1995, 377, 530-532). By this, the sensation of hunger decreases, and the basal metabolic rate within the body rises. Finally, a loss of weight occurs, primarily the body fat rate decreases (M. A. Pelleymounter et al., Science 1995, 269, 540-543, and J. L. Halaas et al., Science 1995, 269, 543-546).

Since the discovery of leptin in 1994, this hormone has always been considered to be also important in the field of reproduction. In the mouse model, female animals which do not produce leptin are infertile (A. M. Ingalls et al., J. Hered 1950, 41, 317-318). However, if they are supplied with leptin, they develop anatomically and functionally normal reproductive organs (F. F. Chehab et al., Nature Genet. 1996, 12, 318-320, and K. Mounzih et al., Endocrinol. 1997, 138, 1190-1193). A normal cycle is developed so that ovulation and fertilization may occur regularly. Pregnancies are carried to full term again without any problems. To date it is not known to which extent leptin influences the reproductive organs directly, or indirectly via the hypothalamo-hypophyseal axis. Likewise, the importance of leptin for the reproduction of humans still is completely unclear. Receptor OB-R for leptin was first detected in the ovary in 1996 by Northern blot analysis (J. A. Cioffi et al., Nature med. 1996, 2, 585-589), whereupon a possible importance of this hormone for the reproduction of humans was postulated. Later on, the short leptin receptor, OB-R_{S}, was found in granulosa and cumulus cells. Both, leptin and leptin-m-RNA, could be detected in granulosa and cumulus cells of the ovary by means of RT-PCR analysis and immunofluorescence. In contrast, in mature human oocytes merely leptin, yet not leptin-m-RNA, was found (J. A. Cioffi et al., Mol. Hum. Reprod. 1997, 3, 467-472). It has been postulated that leptin, which is produced in the granulosa cells of the ovary, is pinocytotically taken up into the oocyte, and therein is peripherally and polarizedly enriched in certain regions. After fertilization and a further development, leptin is found in some cells of the pre-implantation embryo localized together with STAT 3, a key protein of intracellular signal transmission and transcription. It is assumed that leptin is involved in the activation of this transcritpional factor and thus influences the embryonic gene expression (M. Antzcak et al., Mol. Hum. Reprod. 1997, 3, 1067-1086). Another important physiological function of leptin is based on its effect on the steroid hormone synthesis of the ovary, with high leptin concentrations causing an inhibition of the 17β-estradiol synthesis in granulosa cells and thus being capable of impairing the normal function of the ovary (R. J. Zachow et al., Endocrinol. 1997, 138(2), 847-850). In patients afflicted with polycystic ovary syndrome (PCO), infertility and overweight have been functionally and causally associated with increased leptin levels (D. Micic et al., Gynecol. Endocrinol. 1997, 11(5), 315-320).

In WO 98/33865 screening methods for illnesses, in particular cancers, have been suggested by measuring leptin derived from non-fatty tissue. Furthermore, it has also been realized in this WO publication that the amount of leptin in the plasma of pregnant women is markedly increased as compared to the amount which was to be expected on the basis of the body mass index (BMI) of non-pregnant women, exhibiting levels approximately corresponding to the leptin amount in the plasma of overweight subjects. Accordingly, a pregnancy test is suggested in which samples of a non-fatty tissue are taken from pregnant women who are between 8 and 36 weeks pregnant, and the leptin amount contained therein is determined. Then the leptin content determined is compared to the leptin content of a non-pregnant woman of the same age and the same body mass index so as to establish the presence of a pregnancy.

Particularly in the field of reproduction medicine it is necessary to obtain data concerning the fertility, e.g. for the chances and prospects of success of an embryo transfer, even before the pregnancy proper. Moreover, there is a demand for a method by which the period of time of an assisted reproduction-medical treatment can be monitored.

Thus, the present invention has as its object to provide a completely novel fertility determination method by which—apart from a “natural” fertility determination within the scope of a normal cycle—i.a. these data can be established and such monitoring will be ensured.

According to the invention, this object is achieved by a method of determining the fertility of mammals, in particular humans, which is characterized in that

a body or organ fluid is taken from a mammal,

the leptin content is determined in this body or organ fluid, and

the determined leptin content is compared with a reference value so as to determine the fertility.

The present invention is based on the surprising finding that the leptin concentration in the various body or organ fluids where leptin is present directly correlates with the fertility properties. This correlation is not merely restricted to the presence of fertility disturbances, but is also possible for diagnosing fertility fluctuations or for checking the event of the nesting of an embryo in the course of an in-vitro fertilization. According to the invention, it has even been shown that, apart from the detection of the presence or absence of oocytes via the methodology according to the invention for determining the leptin content, even an evaluation of the degree of maturity of oocytes is possible.

In contrast to the pregnancy test according to WO 98/55865, the present invention thus is based on the finding that the property of fertility (and not the pregnancy) directly correlates with the leptin content, thus starting at a much earlier time than does the method according to WO 98/55865: Whereas the pregnancy determination according to WO 98/55865 is described to be possible starting from the 8^{th} week of pregnancy, the test according to the invention starts already long before the beginning of a pregnancy, e.g. within the scope of a normal monitoring of the cycle or an assisted reproduction medication, and ends with the successful implantation of the embryo (i.e. 0^{th}, 1^{st} or 2^{nd} week of pregnancy).

The present invention may be used in human medicine, in particular in the monitoring of in-vitro fertilizations or in fertility diagnoses and expert opinions. Yet it also has enormous possibilities within the scope of modern animal breeding, since it is easy to standardize and does not require complicated laboratory equipment for carrying out the test.

Leptin is present in many different body or organ fluids, and it has been shown according to the invention that the leptin content in all these fluids correlates with the fertility properties. According to preferred embodiments of the present invention, however, the leptin content is primarily determined in body or organ fluids which are characterized by a high physiological leptin content, such as, e.g., serum, follicular or seminal fluid. Yet it is, of course, also possible to carry out the method according to the invention with other body or organ fluids, such as cerebrospinal fluid, since the concentration of leptin in these other fluids also lies in a range which, as a rule, does not cause any problems in terms of the possible leptin detection limit.

In practice, the method according to the invention may be carried out in any manner desired, primarily as regards the manner of taking the body or organ fluid or as regards the determination of the leptin content. The leptin content may, e.g. be determined immunologically, electrophoretically or chromatographically. According to the invention, immunological determination methods often are preferred for leptin, since not only a series of different monoclonal antibodies are available against different epitopes of leptin, but because it is particularly immunological tests, such as in the form of standard ELISA tests, which are easy to design such that they can also be carried out and evaluated without any complex laboratory instruments (e.g. in combination with colorimetric detection methods). In this manner it is possible to provide the determination method according to the invention also in a form which is easy to carry out for common people. Preferably, according to the invention free leptin is determined in the sample.

As the reference value, usually a normal leptin value for the respective body or organ fluid is used. In the present method, this may, e.g., be obtained in the form of comparative values, comparative curves or comparative tables or—as is generally preferred—by a simultaneous determination of a reference sample (having a defined leptin content) together with the sample of the body or organ fluid taken. In the latter instance, the systematic error possibly resulting from using different determination methods or different determination conditions, which probably can never be quite completely eliminated, is avoided right from the beginning. This may be particularly important in determinations where merely gradual differences in leptin contents (e.g. when determining the degree of maturity of oocytes) are at issue.

Preferably, the sample measured according to the invention is not only compared with one reference value, but it is compared with two or more reference values. Thus it is, e.g., possible to provide a different reference value or a reference sample, such as, e.g., a “pathological” reference or a “pregnancy” reference, etc., besides a “normal value”.

According to the invention, however, it is preferred to provide a reference value for the leptin content in the corresponding body or organ fluid which corresponds to the leptin content of a normal patient (or, in animal breeding, to the sample of the normal animal).

When carrying out the method according to the invention, this reference value preferably is obtained in that the leptin content of a reference sample is determined in parallel with the fertility determination of the sample.

According to the invention, the determination of the leptin content preferably is effected by using immunological methods, in particular by using a monoclonal antibody, since by this standardizing can be achieved very efficiently and also for the most varying lots of a determination kit, the data found will be compatible among themselves.

In contrast to the pregnancy test, just like also according to WO 98/55865, in which a pre-implantation development cannot be checked or verified, but only after approximately 3 weeks (with the beginning of a pregnancy merely being estimated due to the missing of the last period), with the test according to the invention the successful nesting process can be verified already at an earlier stage, i.e. already in the 0^{th}, 1^{st} or 2^{nd} week. According to the invention, this verification is already exact, whereas with a pregnancy test on the basis of the above-indicated estimation, always one week too late is estimated (which then is corrected by ultrasonic examinations). Such a correction is no longer required with the method according to the invention, since here the time of implantation of the embryo or of the embryo transfer is known.

In a further aspect, the present invention relates to the use of leptin for determining the property of fertility of mammals.

A further aspect of the present invention relates to a kit for determining the fertility of mammals, which comprises

a sample of a body or organ fluid from a mammal, or a vessel for receiving the body or organ fluid,

a reagent for detecting the leptin content in the sample, and

leptin reference means.

As mentioned above, the choice of the reagent for detecting the leptin content will, of course, depend on the respective detection technique used. For instance, the reagent for detecting the leptin content preferably comprises an antibody to leptin, in particular a monoclonal leptin antibody. Preferably, this leptin antibody also comprises further detection means, such as fluorescent, radioactive or chromogenic groups, or it may be bound by other detection means (e.g., by secondary antibodies).

The leptin reference means preferably comprise a standardized amount of leptin, such as a reference sample of the respective body or organ fluid. On the other hand, the leptin reference means may, however, also consist in a simple comparative value or a comparative table or a comparative curve, respectively, which preferably is standardized for the respective body or organ fluid and the respective detection methodology.

A kit according to the invention is particularly preferred which comprises a whole series of standardized leptin samples, which define a straight calibration line, e.g., or which are representative of certain fertility characteristics.

The method according to the invention, and the kit according to the invention, respectively, preferably are used for monitoring patients within the scope of fertilization methods, in particular in case of in vitro fertilization and intracytoplasmatic sperm injection. For a successful fertilization it is necessary to monitor very precisely the presence or absence of oocytes as well as their quality in the test persons, and to routinely monitor the sperm functionality, respectively, with a simple test.

When using the present invention within the scope of an assisted reproduction-medical treatment, particularly the monitoring of the period of time from the down-regulation to the checking of the successful embryo nesting is essential. This period of time (down-regulation-stimulation-puncture-embryo culture-embryo transfer into the uterus-nesting of the embryo) can reliably and clearly be checked and observed with the method according to the invention (i.e., approximately 4 weeks before to 2 weeks after the embryo transfer).

With this, a predicative and prognostic monitoring, and a predicative and prognostic marker for a diagnosis in the course of an in-vitro fertilization program has been possible and provided, respectively, for the first time. In particular, with the present invention also a prognosis regarding the chances of a success can be made already during the hormonal stimulation procedure.

Accordingly, the method of the invention is applicable much earlier than the pregnancy test described in WO 98/55865, beginning with a completely different starting point, i.e. that of the fertility test. In particular, within the scope of the in-vitro fertilization monitoring according to the invention, it is also precisely known at what time the embryo is transferred, whereas in a pregnancy test this is unknown.

According to a further aspect, also the fertility in case of spontaneous cycles or irregular cycles can be determined with the present invention. Preferably, in case of the fertilization, primarily the data of the normal cycle are used as a basis.

A further preferred use of the test according to the invention or of the kit according to the invention, respectively, resides in determining the degree of maturity of oocytes or sperms.

Moreover, the method according to the invention, and the kit according to the invention, respectively, may be used for investigating fertility and reproduction disturbances, and it is particularly well suited for broad screenings and for the systematic examination of large groups of persons.

A further aspect of the present invention resides in the therapeutic use of leptin for supporting the fertility properties. In this instance, leptin is applied, e.g. in the course of the inventive monitoring procedures of an in vitro-fertilization program, if the leptin amounts measured are not considered to be sufficient for a promising course of the program (e.g., if they are more than 10%, in particular more than 30%, below the normal value for a successful development).

Accordingly, the present invention also relates to the use of leptin for producing an agent for enhancing fertility. In doing so, an effective amount of leptin is administered to the human being or animal (mammal) so as to increase the leptin content to the required levels. Of course, this is not only possible in in-vitro fertilizations, but also in the course of monitoring natural fertilizations and pregnancies.

The present invention shall now be explained in more detail by way of the following examples and the drawing figures. Therein,

FIG. 1 shows the leptin concentration in the follicular fluids of follicles of various sizes;

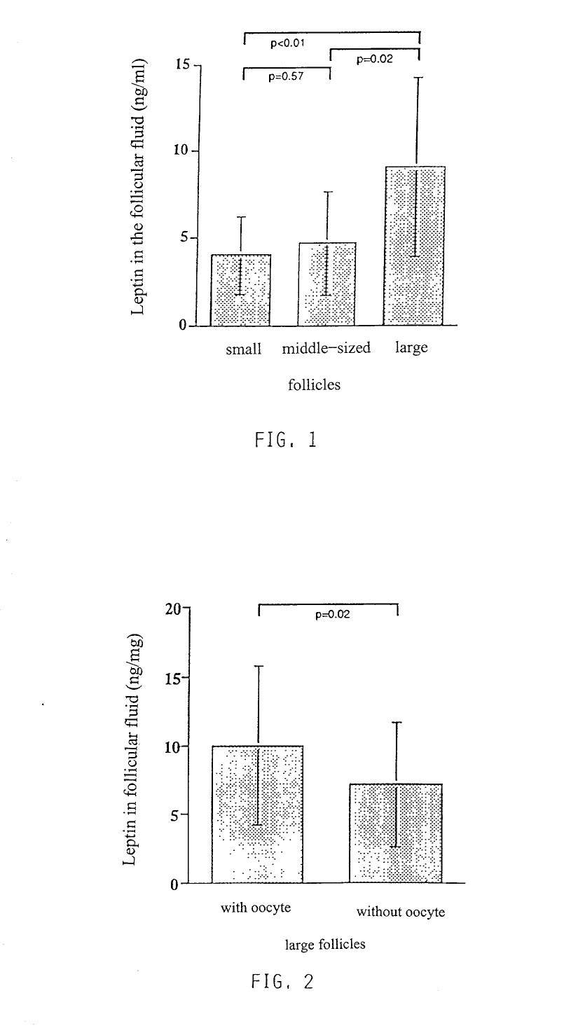

FIG. 2 shows the leptin concentration in follicular fluids with and without an oocyte;

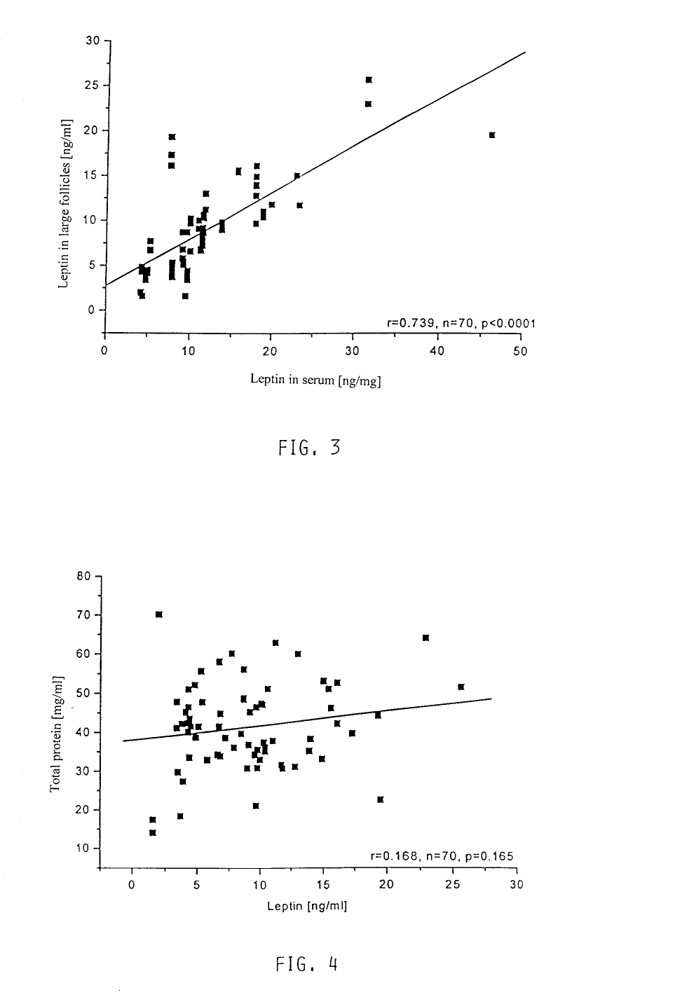

FIG. 3 shows the correlation between the leptin concentration in the follicular fluids of large follicles and in the serum of female patients at the day of follicle puncture;

FIG. 4 shows the correlation between the leptin concentration and the protein content of the follicular fluids of large follicles;

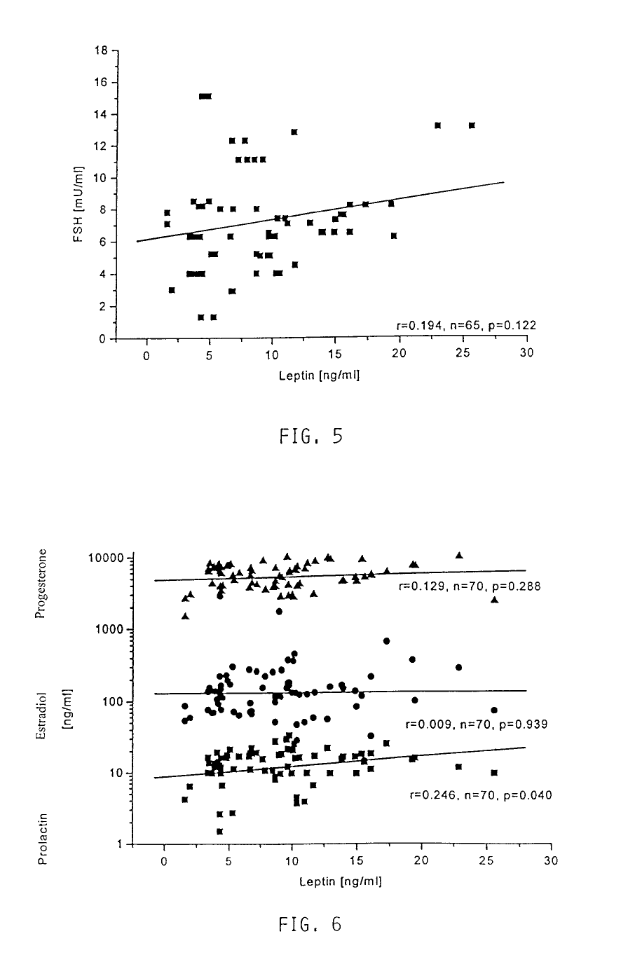

FIG. 5 shows the correlation between the leptin concentration in the follicular fluids of large follicles and the FSH level in the serum after hormonal stimulation with recombinant FSH; and

FIG. 6 shows the correlation between the leptin concentration of estradiol (●), progesterone (▴) and prolactin (▪) in the follicular fluids of large follicles.

### EXAMPLE 1

Follicular fluid and serum

For an IVF or ICSI (intra-cytoplasmatic sperm injection) treatment, 31 female patients were down-regulated on the 21^{st} day of their cycle with leuprorelinacetate (3.75 mg s.c.; Enantone Gyn, Takeda) as GnRH-analogue, and subsequently they were hormonally stimulated with recombinant FSH (Gonal-F, Serono and Puregon, Organon). The respective dose of FSH to be applied was individually adapted by ultrasonic examination and estradiol determination. When at least two follicles had reached a diameter of 20 mm, ovulation was triggered by intramuscular administration of hCG (10,000 IU, Profasi, Serono). Approximately 36 hours after hCG application, the sonographically controlled follicle puncture was effected transvaginally with a subsequent microscopical examination for the presence of oocytes in the aspired follicular fluid. After recovery of the oocytes, the individual follicular fluids were centrifuged, and the supernatants were stored at −196° C. for further determinations. For serum tests, the blood samples drawn at the time of the follicular puncture were centrifuged, and the sera likewise were stored at −196° C.

Leptin determination:

The leptin concentrations were radioimmunometrically determined (Linco Research, St. Charles, Mo., USA), an antiserum without cross-reactions with human insulin, proinsulin, rat insulin, C peptide, glucagon, pancreas-propetide or somatostatin being used. The detection limit was between 0.5 ng/ml and 100 ng/ml.

Hormone determinations:

Estradiol was radioimmunometrically determined (Estradiol MAIA, Bio Chem Immuno Systems, Bologna, Italy). Follicular fluid was diluted 1:100 with the serum of post-menopausal women. The estradiol level of these sera was deducted from the concentration in the follicular fluid.

Progesterone was also radioimmunometrically determined (Orion Diagnostica, Espoo, Finland). Follicular fluid was diluted 1:1000 with the above-mentioned serum. Prolactin was radioimmunometrically determined by means of RIA-Kit (Bio Chem Immuno Systems, Bologna, Italy). Follicular fluids were diluted 1:5 with zero standard to thus have a nearly identical matrix for samples and standards. Measurements of the estradiol, progesterone and prolactin levels were carried out according to the instructions of the product producers. FSH was radioimmunometrically determined (FSH Maioclon, Bio Chem Immuno Systems, Bologna, Italy) in the serum on the day of the puncture.

Protein determination:

The protein amounts in the follicular fluid and in the serum were determined according to the Lowry method (O. H. Lowry et al., J. Biol. Chem. 1951, 193, 265-275), BSA of a known concentration (1 mg/ml) being used as the standard. All the samples were diluted in phosphate-buffered saline (PBS) and measured in the spectrophotometer (Beckman DU 62) at an excitation wave length of 750 nm as compared to the reference value.

Statistics:

Statistical calculations were carried out with the Microsoft Excel Version 5.0 a and Origin V 4.1. Parametrical data were determined by means of the two-tailed t test, correlations were determined by means of the Pearson correlation coefficient, with a p-value of <0.05 being considered as significant.

### RESULTS:

The clinical data of the female patients can be taken from Table 1. Follicular fluids from follicles of various sizes from these patients were examined separately. Moreover, it was taken into consideration whether or not an oocyte was present in the recovered follicular fluid. The leptin concentration in large follicles (ø>19 mm) had a mean of 9.03+/−5.15 ng/ml, in middle-sized follicles (ø 16-19 mm) 4.67+/−2.96 ng/ml, and in small follicles (ø<16 mm) 3.99+/−2.19 ng/ml, the difference in the leptin concentration in large to middle-sized follicles (p=0.02) and small follicles (p<0.01) being statistically significant (FIG. 1). In large follicles with an oocyte, the leptin concentration in the follicular fluid was higher than in large follicles without oocyte (10.12+/−5.67 ng/ml vs. 7.84+/−4.28 ng/ml, p=0.07) (FIG. 2). In the serum of the female patients leptin on the day of follicular puncture was approximately twice as high as the standard levels indicated for unstimulated women (12.68+/−8.98 ng/ml vs. 5.5 ng/ml, cf. C. Schubring et al., J. Clin. Endocrinol. Metabol. 1997, 82, 1480-1483). When comparing leptin in the serum with leptin in the follicular fluid, a statistically highly significant correlation (r=0.74, p<0.0001) was found between the two parameters (FIG. 3). To determine whether or not the leptin content in the follicular fluid possibly is connected with the generally increased protein biosynthesis, the total protein content of the follicular fluids was determined. In doing so, no correlation between leptin and the protein level could be found (r=0.168, p=0.165) (FIG. 4). To discover a possible hormonal regulation of the leptin production under a stimulation treatment, the applied amount of recombinant FSH was compared with the leptin level both in the serum and in the follicular fluid in a study. This showed that no correlation could be established between these two parameters (FIG. 5). Furthermore, estradiol, progesterone and prolactin were determined in the serum and in the follicular fluids. With an increasing diameter of the follicles, an increase in the concentration of these three hormones was found (Table 1). Moreover, it was found that in follicles including an oocyte, the respective hormone values were higher in the punctured follicular fluid than in those without an oocyte (Table 1). However, the hormone values measured in the individual follicular fluids did not correlate with the respective leptin concentrations (FIG. 5). Likewise, no correlation could be found between estradiol (r=0.063, p=0.60), progesterone (r=0.058, p=0.63) and prolactin (r=0.172, p=0.15) in the serum and the respective leptin values in the follicular fluids.

Thus, it could clearly be shown that leptin does occur in human follicular fluid and is produced by granulosa cells of the ovary (J. A. Cioffi et al., Mol. Hum. Reprod. 1997, 3, 467-472). It is assumed that part of the leptin gets from the follicular fluid into the mature oocyte and there is stored in the cytoplasma peripherally and in a polarized state (M. Antczak et al., Mol. Hum. Reprod. 1997, 3, 1067-1086). During a controlled hormonal ovary stimulation, female patients exhibit a significant increase of leptin in the serum from days three to nine of the stimulation cycle, which remains constantly elevated until the time ovulation is triggered (T. Strowitzki et al., Gynecol. Endocrinol. 1998, 12(3), 167-169). Marked fluctuations of the leptin amounts in the sera of female patients during the normal menstruation cycle as well as a leptin deficiency in case of oligo- and amenorrhoe signal a further possible physiological role of leptin for human reproduction (D. Macut et al., Gynecol. Endocrinol. 1998, 12(5), 321-326).

It has been found that both the amount of leptin and also the concentration of the hormones measured in the follicular fluid increase with the size and, thus, the degree of maturity of the follicles. However, no correlation could be found between leptin and the respective hormone values (stimulation with recombinant FSH; cocentration of estradiol, progesterone, prolactin), from which it can be concluded that these hormones do not have a direct influence on the leptin amount in the follicular fluid. Also in the serum of female patients examined, the leptin amount showed to be independent both from the amount of recombinant FSH applied and from the concentration of estradiol, progesterone and prolactin. This is in contrast to examinations of women with spontaneous cycle, in whom leptin and progesteron amounts in the serum correlate (L. Hardie et al., Clin. Endocrinol. 1997, 47, 101-106). In addition, in agreement with other authors (J. A. Cioffi et al., Mol. Hum. Reprod. 1997, 3, 467-472) it has been found that after a controlled hormonal stimulation, the female patients examined did not show any correlation between serum leptin and BMI, which is also in contrast to hormonally unstimulated and non-pregnant women in whom there is a highly significant agreement of these two parameters (L. Hardie et al., Clin. Endocrinol. 1997, 47, 101-106).

Yet, a highly significant correlation has been found between the concentration of leptin in the follicular fluid of large follicles and in serum. From this, it can be concluded that under hormonal stimulation, both the systemic and also the ovarial production of leptin is stimulated. If, on the one hand, only the systemic production of leptin were responsible for the enrichment thereof, approximately equal leptin levels should be found in all the follicles of one and the same female patient. Yet this is not the case. If it was a leptin stimulation that was mainly related to the ovary, a much higher leptin value would have to be expected in the follicular fluids than in the serum. Also this interpretation would be in contrast to the present results.

A marked tendency towards higher leptin, estradiol, progesterone and prolactin levels could be observed in follicles with oocyte in the punctured follicular fluid thereof. Furthermore, for all the hormones examined, a continual increase in the concentration with the follicular diameter can be recognized. From these results it can be concluded that leptin as well as estradiol, progesterone and prolactin are of importance for the growth and differentiation of ovary follicles. However, a direct influence of estradiol, progesterone or prolactin on the leptin synthesis cannot be found. It is a fact that under hormonal stimulation, the leptin synthesis within the body is quite clear, yet is subject to a different or additional regulatory mechanism than in the spontaneous cycle. The methodology according to the invention thus can universally be utilized as a further, independent and reliable instrument for a fertility determination.

TABLE 1
DIAMETER OF FOLLICLES

| | small | middle | large | large with | large with- |
|---|---|---|---|---|---|
| Hormone | (<16 mm) | (16-19 mm) | (>19 mm) | oocyte | out oocyte |
| Estradiol | 58+/−22 | 72+/−28 | 224+/−43 | 276+/−67 | 192+/−46 |
| (ng/ml) | | | | | |
| Progester- | 2575+/− | 2348+/− | 5718+/− | 6618+/− | 5139+/− |
| one | 568 | 682 | 1365 | 1587 | 890 |
| (ng/ml) | | | | | |
| Prolac- | 2,8+/−0,8 | 4,4+/− | 14,0+/− | 17,2+/− | 12,1+/− |
| tin (ng/ml) | | 1,3 | 3,7 | 4,0 | 3,1 |
| Leptin | 3,99+/− | 4,67+/− | 9,03+/− | 10,12+/− | 7,84+/− |
| (ng/ml) | 2,19 | 2,96 | 5,15 | 5,67 | 4,28 |

1. A method for determining the fertility of mammals, in particular of humans, characterized in that
a body or organ fluid is taken from a mammal,
the leptin content is determined in this body or organ fluid, and
the determined leptin content is compared with a reference value so as to determine the fertility.

2. A method according to claim 1, characterized in that the body or organ fluid is serum, follicular or seminal fluid.

3. A method according to claim 1 or 2, characterized in that the reference value is the leptin content in the corresponding body or organ fluid of a normal patient.

4. A method according to any one of claims 1 to 3, characterized in that the reference value is determined in parallel to the fertility determination.

5. A method according to any one of claims 1 to 4, characterized in that the leptin content is determined by immunological methods, in particular by using a monoclonal antibody.

6. A method according to any one of claims 1 to 5, characterized in that the content of free leptin is determined.

7. A kit for determining the fertility of mammals, comprising
a sample of a body or organ fluid from a mammal, or a vessel for receiving the body or organ fluid,
a reagent for detecting the leptin content in the sample, and
leptin reference means.

8. A kit according to claim 7, characterized in that the reagent for detecting the leptin content comprises an antibody, in particular a monoclonal antibody.

9. A kit according to claim 7 or 8, characterized in that the leptin reference means comprises a standardized amount of leptin.

10. A kit according to any one of claims 7 to 9, characterized in that the leptin reference means is a series of standardized leptin samples.

11. The use of a method according to any one of claims 1 to 6 or of a kit according to any one of claims 7 to 10 for monitoring patients within the scope of fertilization methods, in particular in in vitro fertilization and intracytoplasmatic sperm injection.

12. The use of a method according to any one of claims 1 to 6 or of a kit according to any one of claims 7 to 10 for determining the degree of maturity of oocytes or sperms.

13. The use of a method according to any one of claims 1 to 6 or of a kit according to any one of claims 7 to 10 for investigating fertility and reproductive disturbances.

14. The use of a method according to any one of claims 1 to 6 or of a kit according to any one of claims 7 to 10 for determining the fertility in spontaneous cycles or in irregular cycles.

15. The use of a method according to any one of claims 1 to 6 or of a kit according to any one of claims 7 to 10 for checking the nesting of an embryo within the scope of an assisted reproduction-medical method.

16. The use of leptin for preparing an agent for improving the fertility.

17. A method for improving the fertility, which comprises administering an effective amount of leptin to a human or a mammal.

18. Leptin as a medicament for improving the fertility.
